(19) United States
(12) Patent Application Publication
(10) Pub. No.: US 2022/0168374 A1
(43) Pub. Date: Jun. 2, 2022

(54) FOOD SUPPLEMENT COMPOSITION WITH FUNCTIONAL ACTIVES IN THE FORM OF A LIPSTICK

(71) Applicant: Viviane SALVETTI, São Carlos (BR)

(72) Inventor: Viviane SALVETTI, São Carlos (BR)

(21) Appl. No.: 17/246,523

(22) Filed: Apr. 30, 2021

(30) Foreign Application Priority Data
Dec. 2, 2020 (CN) 202011389446.X

(51) Int. Cl. A61K 36/24; A23L 33/105; A61K 36/38; A61Q 1/06; A61K 8/9789; A61K 9/08; A61K 47/36; A61K 8/73; A61K 47/14; A61K 47/24; A61K 47/10; A61K 47/26; A61K 47/46; A61K 47/12

(52) U.S. Cl. CPC A61K 36/24; A61K 2800/43; A61K 36/38; A61Q 1/06; A61K 8/9789; A61K 9/08; A61K 47/36; A61K 8/735; A61K 47/14; A61K 47/24; A61K 47/10; A61K 47/26; A61K 47/46; A61K 47/12; A23L 33/105

(57) ABSTRACT

The present invention relates to functional food supplement formulations comprising a) a phase composed of a mixture of nanosome spheres of biotechnological origin added to low molecular weight hyaluronic acid in a concentration of 5 to 10% by weight, b) a phase of plant actives in a concentration of 10 to 30% by weight, in each case based on the total weight of the formulation, and can optionally also include other active principles, auxiliaries and additives, and the use of this formulation in the form of a functional lipstick.

### CROSS-REFERENCE TO RELATED APPLICATION

This application claims priority from the Chinese patent application 202011389446.X filed Dec. 2, 2020, the content of which are incorporated herein in the entirety by reference.

### FIELD OF THE INVENTION

The present invention relates to functional formulations comprising a) a phase composed of a mixture of nanosome spheres of biotechnological origin added to low molecular weight hyaluronic acid in a concentration of 5 to 10% by weight, b) a vegetable extract phase in a concentration of 10 to 30% by weight, in each case based on the total weight of the formulation, and can optionally also include other auxiliaries and additives, and to the use of this formulation in the form of liquid lipstick as a food supplement, weight loss assistant and cellulite control agent.

The present invention also relates to functional formulations comprising a) a phase composed of a mixture of nanosome spheres of biotechnological origin added to low molecular weight hyaluronic acid in a concentration of 5 to 10% by weight, b) a phase of actives consisting of chromium picolinate, 100 mg, green tea, 50 g, Garcinea cambodia, 150 g, taurine, 50 g, and Fucus 40 g, the active phase being in a concentration of 10 to 30% by weight, in each case based in the total weight of the formulation, and can optionally also include other auxiliaries and additives, and to the use of this formulation in the form of liquid lipstick as a food supplement, weight loss assistant and cellulite control agent.

The present invention also relates to functional formulations comprising deionized water, Klucel HXF, xanthan gum, alginate, picolinate, thryptophane, molibdenium, beetroot, capsico, Nipagim, Nipasol, glycerin, soy lecithin, polyssorbate 80, citric acid, ascorbic acid, sucralose, strawberry flavoring, carmine, Pomceau R4, Bordeaux S and red pigment 40, and can optionally also include other auxiliaries and additives, and to the use of this formulation in the form of liquid lipstick as a food supplement, weight loss assistant and cellulite control agent.

The composition is, ultimately, a liquid food supplement with functional actives, whose appearance resembles a cosmetic such as a lipstick, providing the beautification of the lips, weight loss enhancer and cellulite control agent.

The composition may also comprise natural or synthetic active principles, with medicinal activity, whose appearance resembles a cosmetic such as a lipstick, providing the beautification of the lips, as well as wellbeing.

### BACKGROUND OF THE INVENTION

The desire to look beautiful and healthy has been a human quest for thousands of years. Although the ideal beauty has changed over time, achieving an impeccable exterior appearance has increasingly been a goal of human beings, especially in times of maximum exposure also in the digital world. The term functional cosmetics refers to intermediate products between cosmetics and medicines, which act more deeply on the skin providing greater permeation of the ingredients contained therein. Most of them do not have fragrances or dyes, that is, they are hypoallergenic products, especially indicated for allergic or sensitive people.

Lipstick is one of the most used cosmetics in the world. In addition to usually highlighting the lips, a lipstick can be used for lip care. Like conventional lipstick, lip gloss and contour pencils are also used. However, in addition to its lip-beautifying function, there is a demand to expand the functionality of this cosmetic so present in the daily lives of consumers.

The skin of the lips comprises a thin horny layer and there are no sweat glands on the lips. They are mainly moistened by saliva. In addition, there are only isolated sebaceous glands, so that the surface of the lips is practically free of fat. The lips, therefore, tend to dry out and crack, especially in cold, dry climates. As a general rule, lipsticks are composed of a wax matrix, in which liquid and semi-solid oils, as well as pigments and fillers are incorporated. These anhydrous systems have not, until now, allowed water-soluble constituents to be introduced into the formulations. To remedy this disadvantage of the state of the art, a large number of attempts are made to formulate water-in-oil (W/O) emulsions in the form of a stick. As a general rule, in these cases, relatively large amounts of emulsifier should be used to obtain a stable emulsion and pigment dispersion, as disclosed in EP1018332 and EP1064908.

Document CN108578278 discloses and claims an edible lipstick that can help you lose weight and a method of preparing it. The lipstick is prepared from: 300-500 parts of carnauba wax, 200-400 parts of candelilla wax, 50-80 parts of olive oil, 50-80 parts of sweet almond oil, 50-80 parts of sunflower seed oil, 50-80 parts of grape seed oil, 50-80 parts of shea butter, 100-150 parts of natural vitamin E, 150-200 parts of beeswax and red sorghum pigments. The purpose of this invention is to use pure natural raw materials, and uses the properties of octacosanol (extracted from a plant native to Japan) which, after entering the body, can help to speed up lipid metabolism, reduce lipid accumulation and achieve a good shaping effect.

Document CN110693766, on the other hand, discloses and claims a lipstick containing beta-carotene and a method of preparing healthy cosmetics for lipstick containing beta-carotene. Healthy lipsticks containing beta-carotene comprise the following raw materials in parts by weight of 10 to 20 parts of octyldodecanol, 7.5 to 15 parts of hydrogenated coconut oil, 1.5 to 4 parts of polyisobutylene, 0.75 to 1.5 parts of petrolatum, 2.5 to 5 parts of polyethylene, 1-2 parts of glyceryl isostearate, 1-2 parts of isostearic acid, 2.5-5 parts of cholesteryl oleate, 5-10 parts of malate diisostearyl, 1.5-3 parts of synthetic wax, 5-15 parts of synthetic wax, 2.5-6 parts of bis-diglycerylpolyciladipate-2, 5-10 parts of hydrogenated oil, 4-8 parts of oil hydrogenated castor, 5-10 parts beta-carotene, 5-10 parts tocopherol vitamin E, 3-8 parts bihydroxy imidazolidinylurea, 1-2 parts of essence, 0.4-2 parts of tartrazine n ° 4 and 0.8-3 parts of CI 16185. Thus, an objective of the present invention is to formulate food supplements in liquid form for topical application on lips, with functional hydration and food supplementation properties, containing a mixture of nanosome spheres of biotechnological origin added to low weight hyaluronic acid molecular that promotes softer, smoother and more luminous skin, together with vegetable extracts of gymnema (Gymnema sylvestre R. Br.) and garcinia (Garcinia cambogia L.).

Hydroxycitric acid also has an appetite-reducing action and it is believed that this is due to the alteration of the metabolic flow, resulting from the diversion of carbohydrates from the diet and its metabolites, lipid synthesis. To optimize glycidic metabolism, some authors suggest the association of hydroxycitric acid with chromium picolinate, which acts in the regulation of blood insulin levels.

Tryptophan is an essential amino acid used by the brain, along with vitamin B3, niacin (or niacinamide) and magnesium, to produce serotonin, an important neurotransmitter in the biochemical processes of sleep and mood.

Nutraceuticals are naturally produced and demonstrate that it has a huge effect on the health of people. Whether as a specific ingredient for the body to perform a certain action, or even as a food supplement. Nutraceuticals are pills that represent an extensive range of components that work to help people's health. Regardless of being close to a certain food, they are recognized in a very natural way by the organism that consumes it, as well as what is not necessary the body also eliminates with the same ease.

In addition, it is clear that they are also recognized as functional foods, because they contain a certain substance that is biologically active, it helps the body, benefiting it in some process, such as the metabolic, for example.

In conclusion, so-called nutraceuticals are supplements for food, which have in themselves the concentrated form of so-called CBs, the bioactive compounds out of their original form, and serve to improve or help to recover the health of a person's body.

Gymnema sylvestre, a dicotyledonous medicinal herb belonging to the Asclepiadaceae family, is a woody vine found in tropical Africa, Australia, central and southern India and China. Gymnema is known to have antimicrobial, anti-hypercholesterolemic, and hepatoprotective effects, and in particular, effects on obesity and diabetes mellitus, it has an astringent, stomatal, tonic and refreshing action. It also has a stimulating action on insulin production and diuretic action. More recently, its use has been tried in the treatment of obesity, due to its sweet taste reducing effect, which lasts from 1 to 2 hours, without interfering in the sensation of other flavors such as salty, acidic and astringent. The leaves have a moderate diuretic and laxative activity, due to the presence of flavonoids and anthraquinones.

Garcinia acts on the inhibition of fatty acid synthesis and is a well-known appetite reducer. Assists in pancreatic function and glucose regulation, in reducing serum levels of triglycerides and cholesterol. It also has a laxative action due to the presence of pectins (emollient action). Hydroxycitric acid is an inhibitor of fatty acid synthesis, as it decreases the supply of acetylcoenzyme A in cell metabolism. This occurs by competitive inhibition of the enzyme citratoliase by hydroxycitric acid, since it binds more strongly to the enzyme than citric acid. In addition to inhibiting the synthesis of fatty acids, hydroxycitric acid increases the reduction of fats by reducing levels of malonyl coenzyme A, which activates the enzyme carnitine acetyl transferase. L-carnitine also facilitates the activation of this enzyme, which is why some authors suggest the association of L-carnitine with the extract of Garcinia sp, to promote greater oxidation of fatty acids.

### SUMMARY OF THE INVENTION

Surprisingly, the set of a phase composed of a mixture of nanosome spheres of biotechnological origin added to low molecular weight hyaluronic acid with a vegetal complex phase, in the form of a liquid nutraceutical formulation in the form of a lipstick, showed an excellent sensation of application on the lips in addition to excellent hydration, aid in weight loss and cellulite, with the difference of that lipstick can be used as a food supplement.

The amount of hyaluronic acid decreases in the dermis with age and, thus, this decrease induces deterioration in the quality of the tissue and repair processes and accelerates the skin aging process. As an example, the viscoelastic properties that act as a shock absorber preventing excessive skin rupture (responsible for wrinkles), are reduced and as a consequence the skin becomes aged. There are also consequences on the hydration of cells that lose water and electrolytes that are essential for their life.

The plant extracts phase may comprise extracts of gymnema and garcinia. As previously mentioned, gymnema is a dicotyledonous medicinal herb belonging to the Asclepiadaceae family, known for having antimicrobial, anti-hypercholesterolemic, and hepatoprotective effects, and in particular, effects on obesity and diabetes mellitus, which has an astringent, stomatal, tonic and refreshing. It also has a stimulating action on insulin production and diuretic action. More recently, its use has been tried in the treatment of obesity, due to its sweet taste reducing effect, which lasts from 1 to 2 hours, without interfering in the sensation of other flavors such as salty, acidic and astringent. According to KIM, Hyeon-Jeong et al. Effects of feeding a diet containing Gymnema sylvestre extract: Attenuating progression of obesity in C57BL/6J mice, Asian Pacific journal of tropical medicine, v. 9, n. 5, p. 437-444, 2016, the administration of gymnema extracts in the diet of rats did not produce any change in the blood pressure levels of animals. The dry extract of gymnema (75%) is well tolerated and can be associated with other plants. Garcinia has an extract standardized in 50% hydroxycitric acid. In addition to hydroxycitric acid, it has its isomers I, II, III and IV and anthocyanins: B1 and B2, present in the fruit pericarp; resin, with about 70%, composed of benzophenones and red and yellow xanthones (including morelic acid, isomorelic acid and alpha-gambogic acid) in addition to mucilages, present in the plant. According to ASTELL, K.T. and collaborators A review on botanical species and chemical compounds with appetite suppressing properties for body weight control, Plant Foods Hum Nutr. 2013, 68 (3): 213-21, garcinia hydroxycitric acid is well tolerated.

### DETAILED DESCRIPTION OF THE INVENTION

The functional formulations of the present invention may comprise:

a) a phase composed of a mixture of nanosome spheres of biotechnological origin added to low molecular weight hyaluronic acid in a concentration of 5 to 10% by weight,

b) a plant complex phase in a concentration of 10 to 30% by weight, in each case based on the total weight of the formulation,

it can optionally also include other auxiliaries and additives.

The functional formulations of the present invention may also comprise chrome picolinate, aminoacids, essential minerals and plant extracts in the so-called “plant complex phase”.

Mixtures of nanosome beads of biotechnological origin added to low molecular weight hyaluronic acid are commercially available, the mixture of ethylhexyl palmitate, silica dimethyl silylate; butylene glycol and sodium hyaluronate obtained from BASF under the trade name Epiderfilll being particularly preferred.

Additional ingredients beneficial to the skin and to the body, such as other anti-aging ingredients, vitamins, hormones, pain relievers, anesthetics, botanical extracts, drugs, processing aids, minerals, plant extracts, concentrates of plant extracts, emollients, moisturizers, skin protectors, humectants, silicones, soothing skin ingredients, dyes, perfumes and the like can be added to the formulation. The quantities of such ingredients can be as needed and not limited to any specific limits.

In a preferred embodiment, the functional cosmetic formulation of the present invention comprises the components as listed in table 1.

TABLE 1
components of the preferred embodiment of the present
invention

| COMPONENT | WEIGHT % |
|---|---|
| BASE: polyisobutene, mineral oil, | 50 to 80% |
| hydrogenated ethylene, propylene, | |
| styrene copolymer, hydrogenated | |
| butylene/ethylene, tocopheryl | |
| acetate, ethylhexyl palmitate, | |
| beeswax, STEARETH 2, STEARETH | |
| 20, coconut oil, palm oil, ceresine, | |
| benzophenone-3, saccharin BHT | |
| ANTI-WRINKLE COMPONENT: | 5 to 10% |
| ethylhexyl palmitate, dimethyl silica | |
| silylation, butylene glycol, sodium | |
| hyaluronate | |
| PLANT EXTRACT (e.g. GARCINIA | 5 to 15% |
| CAMBOGIA fruit extract) | |
| PLANT EXTRACT (e.g. GYMNEMA | 5 to 15% |
| SYLVESTRE leaf extract) | |
| COLORANT | 5 to 10% |
| FLAVORING AGENT | 0 to 1% |

In a preferred embodiment, the functional formulation of the present invention comprises the ingredients listed in table 2.

TABLE 2
Components of the formulation

| COMPONENT | WEIGHT |
|---|---|
| Deionized water | 87.849 |
| Klucel HXF | 1.500 |
| Xanthan gum | 0.500 |
| Alginate | 2.000 |
| Picolinate | 2.000 |
| Thryptophane | 0.010 |
| Molibdenium | 0.090 |
| Beetroot | 0.800 |
| Capsico | 0.050 |
| Nipagim | 0.150 |
| Nipasol | 0.200 |
| Glycerin | 2.000 |
| Soy lecithin | 1.300 |
| Polyssorbate 80 | 0.500 |
| Citric acid | 0.150 |
| Ascorbic acid | 0.100 |
| Sucralose | 0.001 |
| Strawberry flavoring | 0.300 |
| Carmine | 0.010 |
| Pomceau R4 | 0.010 |
| Bordeaux S | 0.010 |
| Red pigment 40 | 0.010 |

The invention refers, in addition to the composition itself, to the use of the functional food supplement formulation as a lipstick, in particular as a functional lipstick. The formulation may also act as a plant based medicine, in case specific active principles are added in the so-called plant complex phase.

The invention additionally refers to the use of the functional cosmetic formulation according to the invention as a weight loss and cellulite control agent.

Growing fat lobes push the upper tissues to the skin's surface causing an uneven appearance of the skin known as “orange peel”. Although this phenomenon is physiological at a moderate pace, it is often considered to be a cosmetic and aesthetic problem to be eliminated. That is why the purpose of this certificate of addition is to provide an effective cosmetic solution for reducing measures, capable of visibly combating cellulite and the “orange peel” appearance. In order to answer this, the invention proposes the use of plant extracts of gymnema (Gymnema sylvestre R. Br.) and garcinia (Garcinia cambogia L.).

In fact, surprisingly, a product obtained from plant extracts of gymnema (Gymnema sylvestre R. Br.) and garcinia (Garcinia cambogia L.) has significant effects on the skin with very interesting cosmetic properties, in particular a high lipolytic efficiency and a significant, new and surprising effect on the main fatty acid internalization and metabolism pathways inside adipocytes.

The above description is intended to illustrate the present invention without limiting it. Unless otherwise stated, all amounts of data, content and percentage content are based on the weight and the total amount or the total weight of the formulations.

1. A composition of functional actives in the form of lipstick wherein the composition comprises:
a) a phase composed of a mixture of nanosome spheres of biotechnological origin added to low molecular weight hyaluronic acid in a concentration of 5 to 10% by weight,
b) a plant phase in a concentration of 10 to 30% by weight, in each case based on the total weight of the formulation and can optionally also include other auxiliaries and additives.

2. A composition according to claim 1, wherein phase (a) comprises ethylhexyl palmitate, silica dimethyl silylate; butylene glycol and sodium hyaluronate.

3. A composition according to claim 1, characterized by the fact that the plant extract comprises gymnema e garcinia extracts.

4. A composition according to claim 1, wherein the formulation is composed of: deionized water, Klucel HXF, xanthan gum, alginate, picolinate, thryptophane, molibdenium, beetroot, capsico, Nipagim, Nipasol, glycerin, soy lecithin, polyssorbate 80, citric acid, ascorbic acid, sucralose, strawberry flavoring, carmine, Pomceau R4, Bordeaux S and red pigment 40.

5. A composition according to claim 1, wherein the fact that the formulation is a lipstick.

6. A composition according to claim 5, wherein the fact that it is a lipstick in liquid form.

7. Use of the formulation as defined in claim 1, wherein the fact that it is like a functional lipstick acting on the hydration of the lips.

8. Use of the formulation as defined in claim 1, wherein the fact that it is like a functional lipstick acting as a food supplement.

9. Use of the formulation as defined in claim 1, wherein the fact that it is like a functional lipstick acting on weight loss and cellulite control.

10. A composition according to claim 2, wherein the fact that the plant extract comprises gymnema e garcinia extracts.

11. A composition according to claim 2, wherein the fact that the formulation is composed of: deionized water, Klucel HXF, xanthan gum, alginate, picolinate, thryptophane, molibdenium, beetroot, capsico, Nipagim, Nipasol, glycerin, soy lecithin, polyssorbate 80, citric acid, ascorbic acid, sucralose, strawberry flavoring, carmine, Pomceau R4, Bordeaux S and red pigment 40.

12. A composition according to claim 3, wherein the fact that the formulation is composed of: deionized water, Klucel HXF, xanthan gum, alginate, picolinate, thryptophane, molibdenium, beetroot, capsico, Nipagim, Nipasol, glycerin, soy lecithin, polyssorbate 80, citric acid, ascorbic acid, sucralose, strawberry flavoring, carmine, Pomceau R4, Bordeaux S and red pigment 40.

13. A composition according to claim 2, wherein the fact that the formulation is a lipstick.

14. A composition according to claim 3, wherein the fact that the formulation is a lipstick.

15. A composition according to claim 4, wherein the fact that the formulation is a lipstick.

16. Use of the formulation as defined in claim 2, wherein the fact that it is like a functional lipstick acting on the hydration of the lips.

17. Use of the formulation as defined in claim 3, wherein the fact that it is like a functional lipstick acting on the hydration of the lips.

18. Use of the formulation as defined in claim 4, wherein the fact that it is like a functional lipstick acting on the hydration of the lips.

19. Use of the formulation as defined in claim 5, wherein the fact that it is like a functional lipstick acting on the hydration of the lips.

20. Use of the formulation as defined in claim 6, wherein the fact that it is like a functional lipstick acting on the hydration of the lips.
